(19) United States
(12) Patent Application Publication
(10) Pub. No.: US 2008/0057013 A1
(43) Pub. Date: Mar. 6, 2008

(54) Lip gloss compositions with enhanced shine

(76) Inventors: Padraig Mcdermott, Westfield, NJ (US); Valerie Robert, Charenton Le Pont (FR); Michell Chen, Edison, NJ (US); Ann Marie Rohmeyer, Englishtown, NJ (US); Hy Si Bui, Piscataway, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/514,602

(22) Filed: Aug. 31, 2006

(51) Int. Cl. A61K 8/81; A61K 8/73

(52) U.S. Cl. 424/64; 424/70.15; 424/70.13

(57) ABSTRACT

Disclosed are lip gloss compositions containing a non-polyurethane oil-absorbent particulate material, a film forming copolymer, an oil having a refractive index of at least 1.40, and a wax.

### BACKGROUND OF THE INVENTION

Lip gloss is typically used to enhance natural features by adding color and shine to the lip area. Generally, lip gloss and other lip compositions contain a particulate material such as pigment or particulate fillers in an oil and/or wax base. See, e.g., U.S. Patent Publication 20040161395.

The cosmetic industry has endeavored to provide long lasting lip gloss that retains shine. U.S. Pat. No. 5,747,017 teaches compositions that contain a dimethicone. The compositions taught in U.S. Pat. No. 6,309,629 contain a high weight percentage of gel base consisting of an oil and a copolymer gellant.

U.S. Pat. No. 6,517,823 teaches lip gloss compositions containing at least one glossy film former having a gloss effect measurement of greater than 50%, and at least one thickener and at least one wax present in a combined amount such that the gloss effect measurement of the composition is greater than 50%.

U.S. Patent Publication 20030095936 discloses lip gloss compositions containing a gel/cream base incorporating a mixture of canola oil, Zea mays (corn) starch and silica and two botanicals (vegelatum clear and virgin prunus oil) as a synergistic complex to aid in moisturization.

U.S. Patent Publication 20060134035 discloses cosmetic compositions, including lip gloss compositions, with a long-wear and glossy effect, that contain hydrogenated polycyclopentadiene, and a liquid gloss agent having a refractive index of at least 1.47, such as phenylpropyldimethylsiloxysilicate, polybutene, polyisobutene and hydrogenated polyisobutene.

U.S. Patent Publication 20050276770 to L'Oréal teaches cosmetic products, including lipstick and lip gloss compositions, containing as filler, polyurethane particles and less than 15% by weight of at least one of water and a water-soluble solvent, and that have a mean gloss of at least 30. According to these applicants, the addition of fillers has a detrimental effect on gloss and migration. They discovered that a particular type of filler, namely polyurethane particles, advantageously and unexpectedly allows production of lip care compositions that are satisfactory from the standpoints of gloss and migration.

### SUMMARY OF THE INVENTION

The present Applicants have discovered that other filler materials, namely non-polyurethane oil-absorbent particulate materials, can be added to lip gloss compositions, which achieve excellent gloss that is renewable during wear, and with little or no migration.

A first aspect of the present invention is directed to a lip gloss composition, comprising:

a non-polyurethane particulate oil-absorbent material;

a film forming copolymer in an amount of at least 5% by weight of said composition;

an oil having a refractive index of at least 1.40; and

a wax.

A second aspect of the present invention is directed to a method of making up the lips, comprising: applying a lip gloss composition to lips, wherein the lip gloss comprises a non-polyurethane particulate oil-absorbent material; a film-forming copolymer, an oil having a refractive index of at least 1.4, and a wax.

A third aspect of the present invention is directed to a container containing the lip gloss composition. The container may also contain an applicator such as a brush, or the container may also be configured so as to allow for direct application of the lip gloss to the lips.

Inventive lip gloss compositions provide several advantages. They spread and flatten substantially evenly on the lips. They show little or no bleeding or feathering with wear. They exhibit excellent shine or high gloss, and are renewable or refreshable simply by pursing or pressing the lips together.

### DETAILED DESCRIPTION

The non-polyurethane particulate oil-absorbent materials useful in the present invention absorb oils in an amount of at least a fraction of their weight, and upon compression—such as by pursing or pressing together of the lips—release oil, resulting in a renewable and sustained high shine or gloss effect. Thus, they include particulate materials that have a fillable void or a hollow interior. The term “particulate”, as used herein, is not limited to any particular shape or size. Thus, for example, the particles of the absorbent material may be substantially spherical in nature, but they may be differently shaped (e.g., in the form of platelets or in an elongated manner), or they may be irregularly shaped. Examples of suitable materials include dextrin palmitate, silicone elastomers and styrene/divinylbenzene copolymers. Yet other suitable absorbent materials include expanded powders such as hollow microspheres (e.g., commercially available from Kemanord Plast under the tradename Expancel, and from Matsumoto under the tradename Micropearl F 80 ED.

Exemplary particulate absorbent materials include hydrophobic starches, which are known in the art as starches chemically modified with hydrophobic groups to impart a measure of hydrophobicity to starch (which, in its unmodified state is hydrophilic). Common forms of hydrophobic starches are starch esters containing hydrophobic groups and complex ethers of starch. Starch esters suitable for use in the present invention include the reaction products of cosmetically acceptable polysaccharide acid-esters of substituted dicarboxylic acids of the formula: polysaccharide-OOC—R(R′)—COOH, wherein R represents a dimethylene or trimethylene radical, and R′, which is the hydrophobic group, represents a C1-C18 hydrocarbon substituent group which may be alkyl, alkenyl, aralkyl and aralkenyl, with a di-, tri- or tetravalent cosmetically acceptable metallic or alkaline earth metal ion, e.g., aluminum and calcium. The polysaccharide is typically starch. Dextrin is another example of a cosmetically acceptable polysaccharide. The substituted dicarboxylic acid is typically succinic or glutaric acid. Preferred R′ groups include C8-C12 alkenyl, e.g., octenyl, nonenyl and decenyl. These hydrophobic polysaccharides (and methods of making them, which entail production of the polysaccharide starch-ester by reaction of the polysaccharide and a cyclic dicarboxylic acid anhydride e.g., succinic acid anhydride or glutaric acid anhydride, substituted with the R′ group) are taught in U.S. Pat. Nos. 2,613,206 and 2,661,349. In some embodiments, the hydrophobic starch is an aluminum salt of a starch ester of succinic acid substituted with an octenyl group, known in the art as aluminum starch octenyl succinate. These hydrophobic starches, including aluminum starch octenyl succinate, are commercially available from National Starch and Chemical Co. under the tradenames Natrosorb W and Natrosorb HFW, DryFlo, e.g., DryFlo PC, DryFlo plus and DryFlo AF pure.

The non-polyurethane particulate oil-absorbent material is present in the lip gloss compositions of the present invention in amounts generally ranging up to about 10%, and in some embodiments from about 2% to about 4% by weight of the composition.

Representative oil soluble film forming copolymers that may be useful in the practice of the present invention include the following copolymers: vinylpyrrolidone/vinyl acetate copolymers in which the monomer ratios are from 70/30 to 30/70; vinyl acetate/unsaturated carboxylic acid copolymers such as a copolymer containing 90% of vinyl acetate and 10% of crotonic acid; terpolymers of methyl methacrylate/stearyl methacrylate/dimethylaminoethyl methacrylate, completely quaternised with dimethyl sulphate, the monomers being used particularly in the ratio 20/23/57; and a terpolymer of vinyl acetate/allyl stearate/allyloxyacetic acid, especially in the ratio of 80/15/5; maleic anhydride/methyl vinyl ether copolymers such as those commercially referred to as “Gantrez AN” as well as the ethyl, isopropyl and butyl esters of these copolymers, and maleic anhydride/butyl vinyl ether copolymers.

In some embodiments, the film forming copolymer is an oil (at ambient temperature). Representative examples of such polymers include vinyl pyrrolidone/hexadecene and VP/eicosene copolymers (e.g., commercially available from ISP under the tradename GANEX).

The amount of copolymer present in the lip gloss composition is at least 5% by weight, and in certain embodiments, may be present in an amount of 10%, 15%, 20%, 25%, 30%, 35% or 40% by weight, or higher.

The oil having a refractive index (RI) of at least 1.40 constitutes all or the majority portion, and in some embodiments, the substantial majority portion of a liquid fatty phase of the lip gloss compositions. As disclosed herein, the term “liquid fatty phase” refers to a non-aqueous medium that is liquid at room temperature (25° C.) and atmospheric pressure (760 mmHg, i.e., 105 Pa). The oil may be polar or non-polar (apolar). Examples of oils having a refractive index (RI) of at least 1.40 and which may be useful in the present invention include hydrocarbon-based oils, such as squalene, linear and branched hydrocarbons such as liquid paraffin, liquid petroleum jelly and naphthalene oil, isoeicosane, squalane, polyolefins (e.g., polydecenes such as Puresyn™ 10, polybutene, polyisobutene and mixtures thereof such as Indopol™ L-14), and hydrogenated and partially hydrogenated polyolefins (e.g., hydrogenated polybutene, hydrogenated polydecene, hydrogenated polyisobutene), branched-chain fatty acid esters of glycerol or sorbitol such as polyglyceryl isostearate, polyglyceryl-2 triisostearate, glyceryl triisostearate, and glyceryl tris(2-decyl)tetradecanoate, short chain alkyl or aryl esters (C1-C6) of C12-C22 diacids or diols optionally substituted in available positions by OH, e.g., diisopropyl dimer dilinoleate, C12-C22 alkyl and alkenyl alcohols, long chain alkyl or aryl esters (C8-C36) of C12-C22 diacids or diols optionally substituted in available positions by —OH, e.g., diisostearyl dimer dilinoleate.

Yet other examples of oils useful in the present invention include pentaerythrityl tetrapelargonate, tridecyl trimellitate, triisoarachidyl citrate, pentaerythrityl tetraisononanoate, pentaerythrityl tetraisostearate, phenyl silicones, sesame oil, bis-diglyceryl polyacyladipate-2, ditridecyl dimer dilinoleate, dioctyl dimer dilinoleate, dioctyldodecyl stearoyl dimer dilinoleate, dioctyl dodecyl dimer dilinoleate, dicetearyl dimer dilinoleate, DEDM hydantoin dilaurate, dibutyl adipate, di-C12-15 alkyl adipate, dicapryl adipate, dicetyl adipate, diethylene glycol diisononanoate, diethylene glycol dioctanoate/diisononanoate, dihexyl adipate, dihydroxyethyl soyamine dioleate, diisobutyl adipate, diisocetyl adipate, diisodecyl adipate, diisopropyl adipate, diisostearyl adipate, dimethicone copolyol adipate, dimethyl adipate, dioctyl adipate, dioctyldodecyl adipate, dipropyl adipate, ditridecyl adipate, glycereth-7 diisononanoate, glycol dilaurate, glycol dioleate, methyl glucose dioleate, neopentyl glycol dilaurate, PEG-18 castor oil dioleate, PEG-5 oleamide dioleate, pentaerythrityl dioleate, pentaerythrityl isostearate/caprate/caprylate/adipate, pentaerythrityl stearate/caprate/caprylate/adipate, pentaerythrityl stearate/isostearate/adipate/hydroxystearate, propylene glycol dicaproate, propylene glycol diisononanoate, propylene glycol dilaurate, propylene glycol dioleate, pyridoxine dilaurate, sucrose dilaurate, and DISM (diisostearyl malate).

The aformentioned oils generally have molecular weights ranging from about 650 to about 10,000 g/mol, or even about 750 to about 7500 g/mol. Other glossy oils (that in general have an RI of at least 1.4) and that have a relatively high molecular weight may be selected from linear fatty acid esters with a total carbon number ranging from 35 to 70, hydroxylated esters, aromatic esters, esters of fatty alcohols, fatty acids which are branched, or C24-C28, silicone oils, and oils of plant origin.

In certain embodiments, two or more oils (e.g., 2, 3, 4 or 5 oils) having an RI of at least 1.40 are present in the lip gloss composition. Some film forming copolymers useful in the present invention, such as VP/hexadecene, are oils having an RI of at least 1.40. Not counting these ingredients, broadly speaking, the total amount of oil or oils having an RI of at least 1.40 present in the lip gloss generally ranges from about 30%, 40%, 50% or 60% to about 70% by weight of the composition. The amount of oil may even be higher, e.g., up to 75%, 80%, 85% or to 90% by weight of the composition.

The liquid fatty phase may contain other oils, polar or non-polar, volatile or non-volatile, high viscosity or low viscosity and high molecular weight or low molecular weight.

The wax (which constitutes a solid fatty phase of the lip gloss) is typically a soft wax. Soft waxes have a melting point of greater than or equal to 45° C. and less than 70° C., and a needle penetration of greater than 7.5. The needle penetration of waxes is determined according to French standard NF T 60-123 or U.S. ASTM standard D 1321, at a temperature of 25° C. According to these standards, the needle penetration is the measurement of the depth, expressed in tenths of a millimeter, to which a standardized needle weighing 2.5 g, mounted in a mobile assembly weighing 97.5 g and placed on the wax to be tested, for 5 seconds, penetrates into the wax. Representative examples of soft waxes include beeswax, lanolin waxes, paraffin waxes, spermacetis, certain polyethylene waxes whose molecular weight is such that they satisfy the criteria of melting point and needle penetration, and hydrogenated plant oils (e.g., hydrogenated jojoba waxes and hydrogenated oils which are obtained by catalytic hydrogenation of fatty substances composed of a linear or non-linear C8-C32 fatty chain and which have the qualities corresponding to the definition of the waxes, such as hydrogenated sunflower oil, hydrogenated castor oil, hydrogenated cotton oil, hydrogenated copra oil and hydrogenated lanolin.

In some embodiments, a wax with a higher melting point may also be used, e.g., to adjust viscosity/rigidity of the composition. Examples of such waxes include ceresin waxes, microcrystalline waxes and ozokerites.

The amount of wax generally ranges from about 0.1% to about 15% by weight of the lip gloss composition.

The lip gloss compositions of the present invention may contain a colorant. Colorants may be organic or inorganic in nature, and typically include pigments (including lakes) and dyes (especially liposoluble dyes) usually used in cosmetic or dermatological compositions. The colorant may have any shape, such as, for example, spheroidal, oval, platelet, irregular, and mixtures thereof.

Pigments are generally white or colored, mineral or organic. Representative mineral pigments include titanium dioxide, zirconium oxide or cerium oxide, and also zinc oxide, iron oxide (black, yellow or red) or chromium oxide, manganese violet, ultramarine blue, chromium hydrate and ferric blue and metal powders, for instance, aluminum powder or copper powder. Organic pigments include carbon black, pigments of D & C type and lakes, which are calcium, barium, aluminum or zirconium salts of acidic dyes such as halo acid dyes, azo dyes or anthraquinone dyes. Pigments may be treated so as to make their surface hydrophobic. This treatment may be carried out according to methods known to those skilled in the art. The pigments may especially be coated with silicone compounds such as polydimethylsiloxanes (PDMSs), perfluorinated compounds, lecithin, and amino acids.

The liposoluble dyes include, for example, Sudan Red, D&C Red 17, D&C Green 6, β-carotene, Sudan Brown, D&C Yellow 11, D&C Violet 2, D&C Orange 5 and quinoline yellow.

The colorant e.g., pigment, is typically present in an amount up to 1% by dry weight. In some embodiments, various fillers, although they might not be universally considered as colorants, may be added to provide a coloring effect. Such fillers include nacres (iridescent particles) such as mica coated with titanium oxide, iron oxide, natural pigment or with bismuth oxychloride, and also colored titanium mica. Pearls are another example of a filler that can provide a coloring effect. These fillers may be present in amounts generally ranging from about 1.0% to about 10% by weight of the lip gloss composition.

The lip gloss compositions (may) contain a gelling agent. Suitable agents include non-wax gelling agents such as silicas, silicates, silica silylate and derivatives thereof such as silica dimethyl silylate (commercially available from Degussa under the tradenames Aerosil R 812, R972 and R974). The forementioned gelling agents are hydrophobic. Other suitable gelling agents include polymethylmethacrylate, polyamide powders (e.g., commercially available from Atochem under the tradename Orgasol), acrylate powders such as sodium polyacrylate, and styrene/divinylbenzene copolymers.

The lip gloss compositions may contain a water-absorbent polymer. See, e.g., U.S. Pat. Nos. 6,045,783 and 6,497,891. Examples of water-absorbent polymers include, but are not limited to the following polymers: polymers resulting from polymerization with partial crosslinking of water-soluble ethylenically unsaturated monomers, such as acrylic or vinylic polymers, and such polymers would include crosslinked and neutralized polyacrylates; starch-grafted polyacrylates; acrylamide/acrylic acid copolymers, including sodium salts of such polymers; starch-grafted acrylamide/acrylic acid, including sodium and potassium salts of such polymers; isobutylene/maleic anhydride copolymers; sodium and potassium salts of carboxymethylcellulose; crosslinked salts of polyaspartic acid; and chitosan/polyvinylpyrrolidone and chitosan/polyethyleneimine combinations.

Commercially available water-absorbent polymers include, but are not limited to: crosslinked sodium or potassium polyacrylates, sold under the names SALSORB CL10, SALSORB CL20, “FSA type 101,” and “FSA type 102,” by the company Allied Colloids, ARASORB S-310, from Arakawa Chemical, “ASAP 2000” and ARIDALL 1460, from Chemdal, “KI-gel 201K,” from Siber Hegner, AQUALIC CA W3, AQUALIC CA W7, and AQUALIC CA W10, from Nippon Shokubai, AQUA KEEP D 50, AQUA KEEP D 60, AQUA KEEP D 65, AQUA KEEP S 30, AQUA KEEP S 35, AQUA KEEP S 45, AQUA KEEP A1 M1, and AQUA KEEP A1 M3, from Atochem, and SANWET IM-5000D, from Hoechst Celanese; starch-grafted polyacrylates, sold under the names SANWET IM-100, SANWET IM-3900, and SANWET IM-5000S, from Hoechst; starch-grafted acrylamide/acrylic acid copolymers, in the form of the sodium or potassium salt, sold under the names WATERLOCK A-100, WATERLOCK A-200, WATERLOCK D-200, and WATERLOCK B-204, from Grain Processing Corporation; acrylamide/acrylic acid copolymers, in the form of the sodium salt, sold under the name WATERLOCK G-400, from Grain Processing Corporation; isobutylene/maleic anhydride copolymer, sold under the name “KI Gel-201 K”; carboxymethylcellulose, sold under the name AQUASORB A250, from Aqualon; and chitosan/polyvinylpyrrolidone combinations, sold under the name “Hydrogel AQUATRIX 2,” and chitosan/polyethyleneimine combinations, sold under the name “Hydrogel AQUATRIX 3,” from Hydromer.

The water-absorbent polymer may be present in the lip gloss compositions in an amount ranging from about 0.01 to about 5% by weight, or from about 0.05 to about 3% by weight, or from about 0.1 to about 2% by weight, relative to the total weight of the composition.

In preferred embodiments, the compositions of the present invention are substantially anhydrous (wherein water constitutes no more than about 2% by weight of the composition) or anhydrous (in which case, there is no added water).

The compositions can also contain other cosmetic or dermatologically acceptable adjuvants, which are additional optional ingredients. These ingredients include organic oil soluble sunscreens such as octyl methoxycinnamate, octocrylene and avobenzone, inorganic, e.g., particulate sunscreens such as titanium dioxide and zinc oxide, oil-soluble antioxidants and/or preservatives such as BHT and LIQUAPAR® oil (commercially available from ISP), chelating agents such as disodium EDTA, fragrances (such as pinene), algae, flavoring agents, waterproofing agents, plant extracts and cosmetic active agents, for example, vitamins A, E, C and B3, pro-vitamins, for instance, D-panthenol, calmative active agents, for instance, alpha-bisabolol, aloe vera or allantoin, plant extracts or essential oils, protective or restructuring agents, for instance, ceramides, refreshing active agents, for instance, menthol and its derivatives, moisturizers (arginine PCA), anti-wrinkle active agents and essential fatty acids, and mixtures thereof.

The lip gloss compositions may be formulated by selecting a non-polyurethane particulate material that is oil-absorbent; selecting an oil having a refractive index of at least 1.40; and mixing the oil-absorbent material and the oil, and a film forming copolymer and a wax.

The lip gloss compositions may be applied to lips by hand or with an applicator. A variety of applicators such as brushes, are known in the art. See, e.g., U.S. Pat. Nos. 6,755,585, 5,772,347, 6,607,323 and 6,981,811. In some embodiments, the container is in the form of a flexible (e.g., squeeze) tube (with a closable cap) or a jar (with an accompanying lid), and serves as a reservoir for the lip gloss composition. In these embodiments, the container is packaged alone or together with an applicator such as a brush.

In other embodiments, the container is configured not just as a reservoir, but also to apply or coat the lip gloss compositions on the lips. In particular, the lip gloss compositions may be delivered utilizing a variety of applicators suitable for applying cosmetic compositions, such as a piston-driven applicator. One exemplary applicator may include a reservoir adapted to house the composition. A piston may be adapted to travel through the reservoir upon actuation of an associated piston advancement mechanism to force the composition onto an application surface external to the reservoir through one or more apertures formed through the reservoir. For example, the reservoir may include a first end having one or more apertures beginning inside the reservoir and continuing to an application surface exterior to the reservoir. The reservoir may have an open second end associated with a piston advancement mechanism. A piston may be advanced by the piston advancement mechanism through the open second end to force lip gloss composition housed in the reservoir through the one or more apertures onto the application surface. In such case, the piston is configured to seal against the internal cavity of the reservoir to prevent composition from passing by the piston as the piston is advanced through the reservoir.

In some embodiments, the piston advancement mechanism contains a housing containing a threaded assembly upon which the piston is mounted and an actuation mechanism associated with the threaded assembly. Rotational movement of the actuation mechanism serves to rotate the threaded assembly causing the piston to advance linearly, first through the housing and then through the reservoir, a distance controlled by the thread pitch of the threaded assembly. In other embodiments, the piston is advanced by non-rotational mechanisms, such as a linear mechanism.

In some embodiments, the container is provided with a cover that may be placed over the reservoir and application surface to prevent leakage and contamination. In order to provide such a seal, the cover is preferably sized only slightly larger than the reservoir such that it may fit over the reservoir in a friction fit. The cover may also include one or more internal projections shaped and configured to at least partially enter the one or more apertures of the reservoir when the cover is placed over the reservoir. One or more internal projections may serve to cleanse the apertures and prevent clogging by the composition during each instance in which the cover is placed over the reservoir.

In certain embodiments, the reservoir is (at least partially) transparent such that the lip gloss composition may be seen visually therein. In such cases, the cover may only partially overlap the reservoir such that at least a portion of the composition may be seen visually within the reservoir even when the cover is mounted on the applicator. Such an arrangement provides the user with visual identification of the volume of composition remaining within the reservoir at any given time, regardless of whether the cover is in place or not.

The containers may be suitably packaged e.g., in boxes, blister packages, or they may be shrink wrapped.

The following examples further illustrate the present invention. They are not intended to be limiting in any way. Unless otherwise indicated, all parts are by weight.

### EXAMPLE 1

### Lip Gloss Composition

| Phase | Ingredient | Wt. % |
|---|---|---|
| A | Oil phase | 47.00 |
| | VP/hexadecene | 30.00 |
| | copolymer | |
| B | Waxes | 6.50 |
| C | Pigments | 0.80 |
| D | Pearls | 3.25 |
| E | Aluminum starch | 3.00 |
| | octenylsuccinate | |
| | Silica | 5.25 |
| | Silica dimethyl | 4.00 |
| | silylate | |
| F | Active(s) | 0.10 |
| G | Fragrance | 0.10 |

The oil phase contained the following ingredients, each in a concentration expressed in (% w/w): diisopropyl dimer dilinoleate (9.30), bis-diglyceryl polyacyladipate-2 (26.80), preservatives (1.00), stearyl heptanoate (10.30), polybutene (41.30) and isoeicosane (11.30). These ingredients were combined in a mixing kettle, with heating to 80-85° C., with medium agitation for 15-20 minutes or until homogenous. The lip gloss was then prepared by the following procedure: charge a portion of the oil phase and the VP/hexadecene copolymer (phase A) into the Disconti Mill, with heat to 65° C.; add the pigments (phase C) to make a color phase, and mill for 40-45 min. at 60-65° C., and then check the dispersion; melt the waxes (phase B) a 103° C. in the melting kettle; discharge the color phase from the mill; rinse the mill with the remaining phase A for 20-30 min.; add the color phase into the melting kettle, and heat to 95° C.; using a side sweep blade, mix for 20-30 minutes or until homogeneous; add pearls (phase D), and using a side sweep blade, mix for 20-30 minutes or until homogeneous; add phase E while using a side sweep blade, mix for 20-30 minutes or until homogeneous, while achieving a smooth batch without entrapped air; check color, and using a side sweep blade, mix 20-30 minutes maintaining temperature at 101-103° C.; begin chilling to 25° C., using a side sweep blade, mix for 10-15 minutes or until homogeneous; at 45-50° C., add active(s) (phase F) and fragrance (phase G); mix 5-10 minutes; and drop the batch at 45-47° C. with mixing into appropriate containers.

### EXAMPLE 2

### Rheological Characteristics

Using a controlled stress rheometer, commercially available from TA Instruments under the name AR-G2, the viscosity of the lip gloss of example 1 except without pigment, was measured at 25° C. using a hatched parallel plate having a 40 mm diameter with the gap is set at 1,000 microns. The temperature was precisely controlled by a Peltier system. The viscosity η at a fixed shear rate of 10 s⁻¹ for 10 minutes was 20,000±3,000 cps.

The rheological characteristics of the lip gloss of this example were also studied in the dynamic mode with temperature ramp at 2° C./min through a cycle of cooling and heating from 60° C. to 15° C., and from 15° C. to 45° C. at fixed frequency of 1 rad/s, and 1% strain (in the linear viscoelastic regime). The transition temperature during heating from 15° C. to 45° C. is defined as the temperature at which the elastic modulus G′(ω=1 rad/s)=loss modulus G″(ω=1 rad/s) or

$$tan ( δ ) = \frac{{G}^{″} ( ω = 1 rad \text{/} s )}{{G}^{′} ( ω = 1 rad \text{/} s )} = 1$$

at ω=1 rad/s and 1% strain. The dynamic viscosity is defined as:

$${η}^{′} ( ω ) = \frac{{G}^{″} ( ω )}{ω}$$

The ratio of dynamic viscosity

$$\frac{{⌊ {η}^{′} ( ω = 1 rad \text{/} s ) ⌋}_{T = 20 C}}{{⌊ {η}^{′} ( ω = 1 rad \text{/} s ) ⌋}_{T = 35 C}}$$

at T=20° C. and T=35° C. is defined during heating process. The transition temperature during heating from 15° C. to 45° C. at which the elastic modulus G′(ω=1 rad/s)=loss modulus G″(ω=1 rad/s) at ω=1 rad/s and 1% strain, with a heating rate of 2° C./min, was measured to be in the range of 19° C. to 28° C. More broadly, lip gloss compositions of the present invention exhibit a transition temperature of about 16° C. to 32° C., and in some embodiments, about 18° C. to 30° C. The ratio of dynamic viscosities at 20° C. and 35° C.

$$\frac{{⌊ {η}^{′} ( ω = 1 rad \text{/} s ) ⌋}_{T = 20 C}}{{⌊ {η}^{′} ( ω = 1 rad \text{/} s ) ⌋}_{T = 35 C}}$$

during heating procedure from 15° C. and 45° C. of the lip gloss of this example (i.e., the lip gloss of example 1 but without pigment) was found to be in the range of 30-1500. More broadly, lip gloss compositions of the present invention exhibit a ratio of dynamic viscosities at these temperatures in the range of 10-2000, and in some embodiments, in the range of 20-1800.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A lip gloss composition, comprising:
a non-polyurethane particulate oil-absorbent material;
a film forming copolymer in an amount of at least 5% by weight of said composition;
an oil having a refractive index of at least 1.40; and
a wax.

2. The lip gloss composition of claim 1, wherein said absorbent material comprises a hydrophobic starch.

3. The lip gloss composition of claim 2, wherein said hydrophobic starch comprises a cosmetically acceptable di-, tri- or tetra-valent metal salt of a starch ester of succinic acid substituted with a C8-C12 alkenyl group.

4. The lip gloss composition of claim 3, wherein said hydrophobic starch comprises aluminum starch octenyl succinate.

5. The lip gloss composition of claim 2, wherein said hydrophobic starch is present in an amount of about 2% to about 4% by weight of said composition.

6. The lip gloss composition of claim 1, wherein said film forming copolymer comprises a vinyl pyrrolidone (VP) copolymer.

7. The lip gloss composition of claim 6, wherein said VP copolymer comprises a VP/hexadecene copolymer.

8. The lip gloss composition of claim 6, wherein said VP copolymer is present in an amount of at least about 30% by weight of said composition.

9. The lip gloss composition of claim 1, wherein said oil comprises a polyolefin.

10. The lip gloss composition of claim 9, wherein said polyolefin comprises polybutene, polyisobutene or a mixture thereof.

11. The lip gloss composition of claim 9, wherein said polyolefin comprises a polydecene.

12. The lip gloss composition of claim 1, wherein said oil comprises diisopropyl dimer dilinoleate.

13. The lip gloss composition of claim 1, wherein said oil comprises bis-diglyceryl polyacyladipate-2.

14. The lip gloss composition of claim 1, wherein said oil is present in an amount of at least 30% by weight of said composition.

15. The lip gloss composition of claim 1, further comprising a gelling agent.

16. The lip gloss composition of claim 15, wherein said gelling agent comprises silica, a silicate, silica silylate, silica dimethyl silylate, or mixtures of two or more of said gelling agents.

17. The lip gloss composition of claim 1, further comprising a preservative, filler, or cosmetically acceptable adjuvant, or combination thereof.

18. The lip gloss composition of claim 1, which comprises a polar oil.

19. The lip gloss composition of claim 1, which exhibits a transition temperature in the range of 16° C. to 32° C., as measured during heating from 15° C. to 45° C. and at a heating rate of 2° C./min.

20. The lip gloss composition of claim 19, wherein the transition temperature is in the range of 18° C. to 30° C.

21. The lip gloss composition of claim 19, wherein the transition temperature is in the range of 19° C. to 28° C.

22. The lip gloss composition of claim 1, wherein said wax comprises a soft wax.

23. The lip gloss composition of claim 1, further comprising a water-absorbent polymer.

24. A method of making up the lips, comprising applying a lip gloss composition to lips, wherein said lip gloss comprises a non-polyurethane particulate oil-absorbent material, a film-forming copolymer, an oil having a refractive index of at least 1.4, and a wax.

25. The method of claim 24, wherein the applying is done by applicator.

26. A container comprising a reservoir containing the lip gloss composition of claim 1.

27. The container of claim 26, comprising a jar.

28. The container of claim 26, comprising a flexible tube.

29. The container of claim 26, wherein said reservoir comprises an application surface external to the reservoir and one or more apertures formed through the reservoir, and wherein said container further comprises a piston and a piston advancement mechanism associated with said piston, and wherein the piston is adapted to travel through the reservoir upon actuation of the associated piston advancement mechanism to force the lip gloss composition onto the application surface external to the reservoir through the one or more apertures formed through the reservoir.
